(19) United States
(12) Patent Application Publication
(10) Pub. No.: US 2024/0335701 A1
(43) Pub. Date: Oct. 10, 2024

(54) METHODS AND APPARATUSES FOR ROTATIONAL SUPPORT

(71) Applicant: Motion Therapeutics, Inc., Oakland, CA (US)

(72) Inventors: Cynthia GIBSON-HORN, Oakland, CA (US); James G. PLUEMER, Dana Point, CA (US); Jerome C. HORN, Oakland, CA (US); Katharine Carrie A. CHAMBERLAIN, Oakland, CA (US)

(21) Appl. No.: 18/577,689

(22) PCT Filed: Jul. 8, 2022
(86) PCT No.: PCT/US2022/036582

§ 371 Date: Jan. 8, 2024

Related U.S. Application Data
(60) Provisional application No. 63/219,811, filed on Jul. 8, 2021.

(51) Int. Cl. A63B 23/02; A41C 3/00; A41D 1/089; A61H 23/00; A63B 21/00; A63B 21/065

(52) U.S. Cl. CPC A63B 23/0238; A41C 3/0007; A41D 1/089; A61H 23/00; A63B 21/065; A63B 21/4007; A63B 21/4009; A41D 2400/322; A61H 2201/165; A61H 2205/081; A63B 2213/004

(57) ABSTRACT

Methods and apparatuses (e.g., devices and systems, including garments and software) for improving rotational symmetry in a subject in need thereof. These methods and apparatuses for asymmetrically weighting the subject may engage with the subject's multifidi muscles and therefore adjust for rotational deficit in the subject.

### CLAIM OF PRIORITY

This patent application claims priority to U.S. Provisional Patent Application No. 63/219,811, filed Jul. 8, 2021, and titled “ROTATIONAL SUPPORT FOR CORE STABILITY, BALANCE, AND PERFORMANCE WEAR,” herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### BACKGROUND

Wearable support garments may help promote core stability, enhancing static and dynamic balance and may result in greater strength and coordination, which may in turn help reduce the risk of injury while improving quality of life. For example, U.S. Pat. Nos. 7,156,792, 7,708,673, 10,583,028, and 8,215,773 all described garments and methods of using them to assist in maintaining core stability. Core stability is maintained when the center of gravity is held stable without excessive sway. A person's center of gravity is stable when forces upon the body (from gravity and external ground reaction forces) are absorbed and re-directed by the core musculature, including spinal stabilizers and the pelvic floor. The core musculature works in coordination including spinal and abdominal stabilizers, pelvic floor and diaphragm minimizing disruption to the static or dynamic relationship of the joints within the entire body. The cumulative effect of core stability is synergistic static and dynamic balance within the spine and pelvis, resulting in increased overall strength and coordination, decreasing injury potential and fall risk. Core stability and balance are relevant to individuals of any age to enhance all aspects of daily living, including transition from lying in bed to sitting, sitting to standing, standing, walking, bending, reaching, lifting, workload tolerance, leisure, sport, peak performance, and recovery from movement.

Rotational instability at the spine and pelvis compromises core stability. Instability occurs when the spinal rotators and pelvic stabilizers do not correctly relax or coordinate. Lack of microbalance and coordination amongst these small spinal stabilizing postural muscle groups causes dysfunctional activation patterns resulting in rotational instability of the pelvis and spine. Primary movers (muscles that move the body) take on the stability role; however, they lack attachment to muscles with heightened sensory receptors, resulting in over-delivery of force throughout the body, creating constant chaos of recalibrating efforts through the pelvis and spine searching for a neutral posture and stable center of gravity. Rotational instability can lead to imbalance, potential injury, muscle strain and spasm, soft tissue and joint pain, injury, and overload on both the bony and soft tissue structures resulting in stress injuries and/or increased fall risk.

Previous techniques and apparatuses have sought to provide overall stability of the upper and lower torso via strategic placement of light-weights (e.g., ½, ¼, ⅛, 1/16, 1/32, or less, pounds) placed at a multitude of torso locations based on a perturbation protocol; primarily anterior, posterior, and lateral deficit. However, it would be particularly helpful to provide method and apparatuses that may address the need for improving or enhancing rotational symmetry to facilitate core stability, balance and performance.

### SUMMARY OF THE DISCLOSURE

Described herein are methods and apparatuses (e.g., devices and systems, including garments and software) for improving rotational symmetry in a subject in need thereof. These methods and apparatuses for asymmetrically weighting the subject may engage with the subject's multifidi muscles and therefore adjust for rotational deficit in the subject. For example, described herein are methods of treating subject, the method comprising: determine that the subject has a rotational deficit; and applying an asymmetric stimulus to engage the subject's multifidi muscles on opposite sides of the subject's spine at approximately a same horizontal level.

Applying the asymmetric stimulus may comprise positioning a weight or weights, e.g., so that the asymmetric weight provides a stimulus to the multifidi muscles. Alternatively or additionally, applying the asymmetric stimulus may comprise applying an electrical stimulation and/or a vibrational stimulation. For example, applying the asymmetric stimulus may comprise applying an electrical stimulation and/or a vibrational stimulation in addition to positioning a weight. In some examples, applying the asymmetric stimulus comprises applying a pair of weights symmetrically on opposite sides of the subject's midline at the same horizontal level, further wherein the weights each have a different weight. Applying the asymmetric stimulus may comprise applying the asymmetric stimulus to a posterior aspect of the subject's lower back. As used herein the lower back may include the waist and gluteal region. Applying the asymmetric stimulus may comprise applying the asymmetric stimulus to a region of the subject's body between the subject's first lumbar vertebra (L1) and a bottom of the subject's sacrum level (S5). Alternatively in some examples applying the asymmetric stimulus comprises applying the asymmetric stimulus to the thoracic region (e.g., T1-T8). Applying the asymmetric stimulus may comprise applying the asymmetric stimulus upon or about and between the subject's posterior edges of the subject's iliac crest. In some examples, applying the asymmetric stimulus comprises applying a light weighting to engage the subject's multifidi muscles, wherein the light weighting weights less than 3.5 pounds. In some cases the weight may be greater than 3.5 pounds (e.g., 4 pounds, 5 pounds, etc.).

Any of these methods may also include applying one or more weights (asymmetrically or symmetrically) at other regions of the body.

In any of these methods, determining may comprise detecting one or more of: a rotational weakness, instability, pelvic obliquity, dyskinesia, latent firing of a spinal rotator muscle(s), with one or more maladaptive movement responses. The maladaptive movement may be, e.g., in the lower body, which may include one or more of: pelvis drop, hip internal rotation and/or adduction, knee valgus, lower leg external rotation, ankle and/or foot pronation, great toe or all toes extension. The maladaptive movement may be, e.g., in the upper body, which may include one or more of: rib flare, trunk rotation and/or lateral flexion, scapular protraction and/or elevation, glenohumeral internal rotation, neck extension and/or rotation, forward head posture, asymmetric arm swing during movement. For example, determining may comprise using an application software to determine where to apply the asymmetric stimulus.

Applying the asymmetric stimulus may comprise wearing a pre-weighted garment on the subject's body, such as any of the garments described herein. Alternatively, the garment may be configured to allow adding/removal of the stimulation (e.g., weighting). In some examples the garment may be configured as an undergarment, shorts, belt, shirt, etc. In general, the garment may be a waisted garment, covering the waistline region of the subject.

Any of these methods may include instructing the subject to perform a predetermined series of movements while applying the asymmetric stimulus. For example, these methods may include instructing the subject to exercise while applying the asymmetric stimulus.

In general, these methods may be methods for treating a patient having an issue that is based on a problem with rotational stabilization, or that may benefit from an improved rotational stabilization. In one example in particular, the methods and apparatuses described herein may be for treating a pelvic floor dysfunction. Alternatively, in some examples, these methods may be methods of generally enhancing activities of daily living and/or athletic performance.

For example, a method of treating a subject may include: identifying a rotational deficit about a segment of the subject's spine; and weighting, asymmetrically, a fixed position on either side of the subject's spine at a same level. Weighting may further include applying an electrical stimulation and/or a vibrational stimulation at the fixed position on at least one side of the subject's spine at the same level. Weighting may comprise applying a weight to a posterior aspect of the subject's body. For example, weighting may comprise applying weighting a region of the subject's body between the subject's first lumbar vertebra (L1) and a bottom of the subject's sacrum level (S5). In some examples, weighting comprises applying weighting at or between the subject's posterior edges of the subject's iliac crest. As mentioned above, weighting may comprise applying a light weighting to engage the subject's multifidi muscles. Weighting may comprise applying a light weighting weight less than 5 pounds (e.g., 4 pounds or less, 3.5 pounds or less, 3 pounds or less, 2 pounds or less, 1.5 pounds or less, 1 pound, or less 0.5 pounds or less, 0.25 pounds or less, etc.). Weighting may comprise wearing a pre-weighted garment on the subject's body.

As mentioned above, identifying the rotational deficit about the segment of the subject's spine may comprise detecting one or more of: a rotational weakness, instability, pelvic obliquity, dyskinesia, and latent firing of a spinal rotator muscle, with one or more maladaptive movement responses comprising one or more of: pelvis drop, hip internal rotation and/or adduction, knee valgus, lower leg external rotation, ankle and/or foot pronation, and great toe or all toes extension, in the lower body, and/or one or more of the following: rib flare, trunk rotation and/or lateral flexion, scapular protraction and/or elevation, glenohumeral internal rotation, neck extension and/or rotation, forward head posture, and asymmetric arm swing during movement, in the upper body. In some cases, identifying the rotational deficit about a segment of the subject's spine comprises using an application software to determine where to apply the weighting. The subject may be asked to perform one or more maneuvers that may expose a rotational deficit; the rotational deficit may be automatically or semi-automatically detected (e.g., using software, firmware and/or hardware, including an application software, “app” being executed on a subject or user's personal computer and/or portable computing devices such as a phone, tablet, etc.).

Any of these methods may include instructing the subject to perform a predetermined series of movements while applying the weighting. For example, any of these methods may include instructing the subject to exercise while applying the device and/or system of weighting or external stimulus.

Also described herein is software, firmware, and/or hardware for performing any of these methods. For example, described herein are non-transitory computer-readable medium including contents that are configured to cause one or more processors to perform any of the methods described herein.

Also described herein are garments for treating rotational deficit about one or more segments of a subject's spine, the garment comprising: a body region configured to be worn by the subject; and a first weight and a second weight, wherein the first weight is greater than the second weight, further wherein the first and second weights are symmetrically coupled to the body region and configured to be positioned on the subject's body between either the subject's first thoracic vertebra (T1) and the subject's eighth thoracic vertebra (T8) or between the subject's first lumbar vertebra (L1) and a bottom of the subject's sacrum level (S5) symmetrically on either side of the patient's spine so that the subject is asymmetrically weighted when the garment is worn by the subject.

In general, the body region may be configured to secure the first weight and the second weight against the subject's skin when the garment is worn. For example, the garment may be formed of at least an elastic fabric, a compression fabric, etc. For example, the portion of the garment over the stimulators (e.g., weights, electrical stimulators, vibrational stimulators, etc.) may include a polymeric material (e.g., a polyurethane/rubber material, such as Lycra, Spandex, etc., nylon, neoprene, etc.). The body region may be configured as a two-way or four-way stretch. The body region may be formed of a knit, woven or mesh material.

The first weight and the second weight may be separated from each other by between about 0.1 cm and 20 cm (e.g., between 0.2 cm and 18 cm, between 0.5 cm and 20 cm, between 0.5 cm and 18 cm, between 0.5 cm and 15 cm, etc.).

Any of these garments may be reversible.

For example, the garment may be configured as a pair of shorts, an undergarment, or a belt, wherein the first and second weights are configured to be positioned on the subject's body between the subject's first lumbar vertebra (L1) and the bottom of the subject's sacrum level (S5) symmetrically on either side of the patient's spine when the garment is worn by the subject.

For example, a garment for treating rotational deficit about one or more segments of the subject's spine may include: a waisted region configured to be worn by a subject; and a first weight and a second weight, wherein the first weight is greater than the second weight, further wherein the first and second weights are each coupled to the torso region and configured to be positioned on the subject's body between the subject's first lumbar vertebra (L1) and a bottom of the subject's sacrum level (S5) symmetrically on either side of the patient's spine so that the subject is asymmetrically weighted when the garment is worn by the subject.

Any of these garments may be configured as a bra or an undergarment, wherein the first and second weights are configured to be positioned on the subject's body between the subject's first thoracic vertebra (T1) and the subject's eighth thoracic vertebra (T8) symmetrically on either side of the patient's spine when the garment is worn by the subject.

For example, a weighted garment for treating rotational deficit about one or more segments of the subject's spine may include: a torso region configured to be worn by a subject; and a first weight and a second weight, wherein the first weight is greater than the second weight, further wherein the first and second weights are each coupled to the torso region and configured to be positioned on the subject's body between the subject's first thoracic vertebra (T1) and the subject's eighth thoracic vertebra (T8) symmetrically on either side of the patient's spine so that the subject is asymmetrically weighted when the garment is worn by the subject.

In any of these examples, the first weight and the second weight may be part of a compound weight attached to the garment. For example, the compound weight typically includes a pair of different weights (one lighter than the other) that are fixed to a base at a predetermined distance apparat. In some examples the first and second weights are attached to a base that is configured to removably couple to a garment (e.g., shirt, pants, bra, belt, etc.) by a removable attachment. An example of an attachment may include a hook-and-loop (e.g., Velcro) material. The garment may be configured to allow attachment of the compound weight along the patient's spinal region (e.g., midline of the back). Also described herein are systems or sets of compound weights that include weights of different values and/or different separation distances between the weights.

As mentioned, any of these garments may include an electric stimulator or vibrational stimulator positioned with either the first weight or the second weight, or a first electrical or vibrational stimulator positioned with the first weight and a second electrical or vibrational stimulator positioned with the second weight.

In any of the apparatuses (e.g., garments, apps, etc.) and methods described herein, an electrical and/or vibrational stimulator may be used instead or in addition to the weights. For example, a garment may include a first electrical stimulator and a second electrical stimulator, wherein the first and second stimulators are symmetrically coupled to the body region and configured to be positioned on the subject's body between either the subject's first thoracic vertebra (T1) and the subject's eighth thoracic vertebra (T8) or between the subject's first lumbar vertebra (L1) and a bottom of the subject's sacrum level (S5) symmetrically on either side of the patient's spine. The first electrical stimulator may be configured to apply a greeter intensity of stimulation as compared to the second electrical stimulator. For example, the first electrical stimulator may be configured to apply at or above a sensing threshold for sensing the stimulation while the second electrical stimulator may be configured to apply at below the sensing threshold. The sensing threshold may be determined specific to a particular patient. In some examples the electrical stimulation may be between about 1 mA to 200 mA (e.g., 1 mA to 150 mA, 1 mA to 100 mA, 1 mA to 50 mA, 1 mA to 40 mA, 1 mA to 30 mA, 1 mA to 25 mA, 5 mA to 100 mA, 5 mA to 50 mA, 5 mA to 40 mA, 5 mA to 30 mA, etc.) and 1 to 200 Hz (e.g., 1 to 150, 1 to 100, 1 to 50, 30-200, 30-150, 30-100 Hz, 40-200 Hz, 40-150 Hz, 40-100 Hz, 50-200 Hz, 50-150 Hz, 50-100 Hz, 80-200 Hz, 80-150 Hz, etc.), a pulse width of between about 10 μs to 400 μs (e.g., 10 μs to 300 μs, 10 μs to 250 μs, 10 μs to 200 μs, 40 μs to 300 μs, 40 μs to 250 μs, 40 μs to 200 μs, 100 μs to 400 μs, 100 μs to 350 μs, 100 μs to 300 μs, etc.). Both the first and second electrical stimulators may be configured to apply within these parameter ranges, but may be specifically configured so that the first electrical stimulator applies at different parameters (typically having a greater intensity) than the parameters of the second electrical stimulator. For example the first electrical stimulator may apply stimulation of between about 60-200 mA and the second electrical stimulator may apply stimulation of between about 1-50 mA). The intensity may be ramped up over time.

In some examples a garment may include a first vibrational stimulator and a second vibrational stimulator, wherein the first and second vibrational stimulators are symmetrically coupled to the body region and configured to be positioned on the subject's body between either the subject's first thoracic vertebra (T1) and the subject's eighth thoracic vertebra (T8) or between the subject's first lumbar vertebra (L1) and a bottom of the subject's sacrum level (S5) symmetrically on either side of the patient's spine. The first vibrational stimulator may be configured to apply a greeter intensity of stimulation as compared to the second vibrational stimulator. In some examples the vibrational stimulation may be between about 1-500 Hz (e.g., between about 1-400 Hz, between about 1-350 Hz, between about 1-300 Hz, etc.) and between about 0.1 to 12 mm (e.g., between about 0.12 to 10 mm, etc.).

For example, described herein are weighted garments for treating rotational deficit about one or more segments of the subject's spine, the garment comprising: a waisted region configured to be worn by a subject; and an asymmetric stimulator (e.g., weight) coupled to the waisted region and configured to be positioned on the subject's body between the subject's first lumbar vertebra (L1) and the bottom of the subject's sacrum level (S5) on just one side of the patient's spine on a side of a posterior edges of the subject's iliac crest that is proximate to the subject's spine so that the subject is asymmetrically stimulated (e.g., weighted) when the garment is worn by the subject. The asymmetric stimulus may be a weight and/or may be an electrical stimulation and/a vibrational stimulation and/or a combination of any of these.

These garments may be configured as under garments, shorts, pants, belts, etc. In particular, described herein are reversible garments that may allow asymmetric stimulation (e.g., weighting) more on one side (e.g., the left side) in one configuration, but may be reversed to allow asymmetric stimulation more on the opposite (e.g., right) side.

In some examples the garment may be configured to allow adjustment of (or addition to) the asymmetric stimulation and/or additional stimulation. In some examples the garment may be configured to provide an asymmetric weight that is between about 1/32 and 5 pounds. In some examples the garment may be configured to provide a second weight coupled to the waisted region, wherein the second weight is less than the asymmetric weight and is positioned on opposite side of the subject's spine from the asymmetric weight when the garment is worn by the subject.

Any of these garments may include the asymmetric stimulation. For example the asymmetric stimulation may comprise a pair of separate weights that may be positioned at the same level of the body but on opposite sides of the spine. Alternatively, or additionally, the asymmetric weights may be compound weights that include a pair of weights in which the heavier weight is on one side of the compound weight and the lighter weight is on the other side.

All of the methods and apparatuses described herein, in any combination, are herein contemplated and can be used to achieve the benefits as described herein.

### BRIEF DESCRIPTION OF THE DRAWINGS

A better understanding of the features and advantages of the methods and apparatuses described herein will be obtained by reference to the following detailed description that sets forth illustrative embodiments, and the accompanying drawings of which:

FIG. 1A is a drawing illustrating the spinal anatomy including the layers of the multifidus that provide rotational stability.

FIG. 1B schematically illustrates asymmetric stimulation (e.g., weighting) that may be attached to a garment to be worn by the subject, in this example just on and above the PSIS (Posterior Superior Iliac Spine).

FIGS. 2A-2C illustrate examples of apparatuses (garments) for applying therapeutic asymmetric weighting. In FIG. 2A the garment is a pair of shorts that include an asymmetrically weighted input panel attached to the back. FIG. 2B shows a pair of underwear that includes an asymmetrically weighted input, and FIG. 2C shows an asymmetrically weighted belt that includes a pair of fixed weights of different values.

FIGS. 3A and 3B show another example of a garment as described herein including an asymmetrically weighted waisted region. In FIG. 3A the front view of the garment also includes tensioning materials pulling to align internal obliques and to support rotation and intraabdominal pressure. FIG. 3B shows a side view of the garment of FIG. 3A, showing the asymmetrically applied weight.

FIG. 4 illustrates an example of a garment (e.g., shorts) as described herein including a weighting for the iliopsoas muscle of the inner hip.

FIG. 5 is a side view of a garment (e.g., shorts) as described herein configured to weight the gluteus mediuis.

FIG. 6 is another example of a side view of a garment as described herein including a silicone pad surrounding the greater trochanter.

FIG. 7 is a front view of an example of a garment having an open crotch region as descried herein.

FIGS. 8A and 8B illustrate an example of a garment for asymmetric weighting as described herein. In FIG. 8A the front of the garment (e.g., shirt) is shown including asymmetric weighting both on opposite sides of the spine, but also around the external/internal obliques.

FIG. 8B is a side view.

FIG. 9 is another example of a garment for asymmetric weighting as described herein.

FIGS. 10A-10B illustrate an example of a garment for asymmetric weighting for rotational stabilization as described herein. FIG. 10A shows the front, and FIG. 10B shows the back.

FIG. 11A illustrates one example of a garment (e.g., shorts or undergarment) for asymmetric weighting to assist in rotational stabilization of a subject. FIGS. 11B-11E illustrate examples of weights (individual weights in FIGS. 11B and 11D and compound weights in FIGS. 11C and 11E) that may be used with or as part of the garment to provide asymmetric stimulation (e.g., weighting) to modulate rotational stabilization.

FIG. 12 schematically illustrates one example of a method of treating a patient to improve rotational stabilization as described herein.

### DETAILED DESCRIPTION

The methods and apparatuses described herein are configured to treat subjects (e.g., individuals) experiencing rotational instability and/or identified rotational deficits. The inventors have surprisingly found that treatment of rotational instability and/or identify rotational deficit, even without treatment for other imbalances, may resolve a large variety of neuromuscular disorders. In particular, these methods and apparatuses may provide a light asymmetrical weighting to treat rotational stabilization, improving the body's center of gravity about the spine and affecting all planes of movement. Facilitation of the segmental rotators of the spine may coordinate spinal stabilization with the pelvic musculature. This may allow the primary muscles at the major joints to perform more efficiently with focus on force production for power and movement without the burden of co-contraction of primary muscles to stabilize the pelvis and spine.

Typically, primary movers are for power movements of levers through joints, not for stabilization of joint integrity. The chronic overwork of primary movers in the dual role of joint stability and power generation, specifically in the pelvis, may create a constant concentric shortening and the loss of eccentric control throughout the pelvic floor, anterior hip, and spine. This may result in asymmetrical muscle activation, stiffness and loss of flexibility, complaints of the pain in the lower back and extremities and throughout the body, as the body instinctively seeks out often painful reactive balance solutions. Light facilitation supporting anti-rotational control of the spine and pelvis increases joint integrity throughout the body. Facilitation allows the primary movers to elongate into their resting position and relax and reduce tightness and stiffness complaints, resolving chronic pain and supporting the recovery of movement.

Laxity of the right or left sacroiliac joints has been related to sacroiliac joint pain and asymmetrical tightness of the pelvic floor. Loss of pelvic floor and spinal stabilizer synergy and tone create maladaptive movement responses throughout the body including muscle weakness and/or increased tone. The muscles in the pelvic floor and spinal stabilizers must work in concert to provide stability to the core. Substitution strategies create weakness and asymmetry which may include overrecruiting the pelvic floor and the primary mover muscle groups (quadriceps, hamstrings, gluteals, back extensors rectus abdominous, etc.) often in an asymmetrical pattern. Pelvis floor tightness and weakness can cause pain, pressure, incomplete voiding, incontinence of bowel and bladder, and sexual dysfunction, among other issues. Biofeedback, relaxation, electrical stimulation, and exercises are common in the treatment of the pelvic floor. Lightly weighting the body asymmetrically for rotational symmetry of the pelvis is novel in the treatment of pelvic floor dysfunction. As described herein, coordinated activation and relaxation with resultant stabilization of the pelvis, low back rotators, extensors, and the abdominals may dramatically improve the pelvic floor function. In many of the examples described herein the asymmetric stimulation may be provided by differential weighting to engage the subject's multifidi muscles, however alternative or additional stimulation may be applied. For example, asymmetrical amounts of electrical stimulation (e.g., transcutaneous stimulation) may be applied at different intensities to the muscles instead of or in addition to differential weighting. The combination of such techniques may enhance control and learn more quickly.

Although weighted garments have been described, such garments typically rely on heavily weighting the torso or legs for strength training and their main focus is not the facilitation of rotation for stabilization. The methods and apparatuses described herein typically rely on asymmetric weight positioning, and specifically light asymmetrically weighted clothing for sensory cueing and proprioception to enhance rotational symmetry, extensor stabilization, lower abdominal facilitation, and/or pelvic floor control.

The methods and apparatuses described herein apply strategic placement of light (e.g., 5 pounds or less, 4 pounds or less, 3.5 pounds or less, 3 pounds or less, 2.5 pounds or less, 2 pounds or less, 1.5 pounds or less, 1 pound or less, 0.5 pound or less, etc.) asymmetrical weights upon the multifidi and spinal extensors, providing sensory augmentation cueing for engagement and coordinated facilitation of these specific muscles directly under the lightly asymmetrical weighted input. Providing a sensory cue or input over the multifidi at the pelvis and spinal extensor/stabilizers enhances rotational strength, engagement, and power.

These methods and apparatuses may include the application of pairs of stimulation (e.g., weights) symmetrically positioned on either side of the spine, using asymmetric stimulus (e.g., different weights) at the same level. The level may refer to the spinal level (e.g., T1-T8, L1-bottom of sacrum). Both weights may be part of the same base (e.g., a compound weight) or may be individual weights. The weights may have the same general size and/or shape or may be different sizes and shapes.

Light weighting or input of any kind in an asymmetrical manner may provide sensory input to the multifidi at the lumbar spine levels and the upper and lower sacral area, and may provide rotation, extension, lateral flexion stability. In addition, this stabilizing asymmetric stimulation may immediately improve posterior stability to a posterior perturbation that was not previously known. For example, weight amounts in these locations may be determined by the amount of rotational deficit of the spine and pelvis, or a Trendelenburg moment (a Trendelenburg is defined as the weak side at the pelvis will cause a drop to the contralateral side. For example, when there is a right Trendelenburg, the left pelvis drops because there is too much weakness on the right to maintain a neutral pelvis.) at the hip during single limb activities, rotation and/or anterior lateral deficit upon visual or three-dimension observation using any equipment (video, motion capture, posture analysis in various positions, gyroscopic, accelerometry, etc.), of functional activities on varied surfaces to reveal a deficit. For example, a light weight (e.g., ½ to 1/32^{nd} of a pound) may improve a maladaptive movement response by facilitation of three-dimensional control. In some examples other inputs may also be used to improve control at the specific joints improving overall function. For example, vibration, electrical stimulation or combination of stimulation, including weights may be used. The use of a combination of a symmetrical horizontal weight placement of asymmetric amounts of weight may be particularly effective. For example, a differential weighting strategy may involve placing a heavier weight on one side of the posterior pelvis while a lesser amount of weight may be placed on the contralateral side of the posterior pelvis, providing an asymmetric input to the pelvis and facilitating improved control in a rotationally unstable individual. Although weight is applied on both sides (and in a symmetric location on either side of the spine at the same spinal level), the amount of weight is different (asymmetric). Any asymmetrical stimulation (input) or amount may be used, and light weights may be sufficient for everyday activities. However, overweighting (using heavier weights) may be used for performance enhancement of some athletic activities, which may result in an improved performance during such high-level activities. For instance, using a combination of weighting as mentioned above and increasing the weight asymmetrically or using the combination above and adding weight equally may improve performance above and beyond regular performance.

Without being bound by a particular theory of operation, rotational stability may enhance balance and performance through the nano-sensory proprioceptive influence provided via light touch to the skin, pressure, and stretch to facilitate muscles directly under the weight/sensory input. The multifidi have higher degrees of proprioceptors and attach through the spine and into the sacrum. Specifically, the multifidi connect to spinal segments, sacral segments, and the posterior Iliac spinous process (PSIS), stabilizing the pelvis and lower trunk. The sacral multifidus and connection over the PSIS may be directly related to stabilizing the pelvis as well as stabilizing the forces on the pelvic floor muscles.

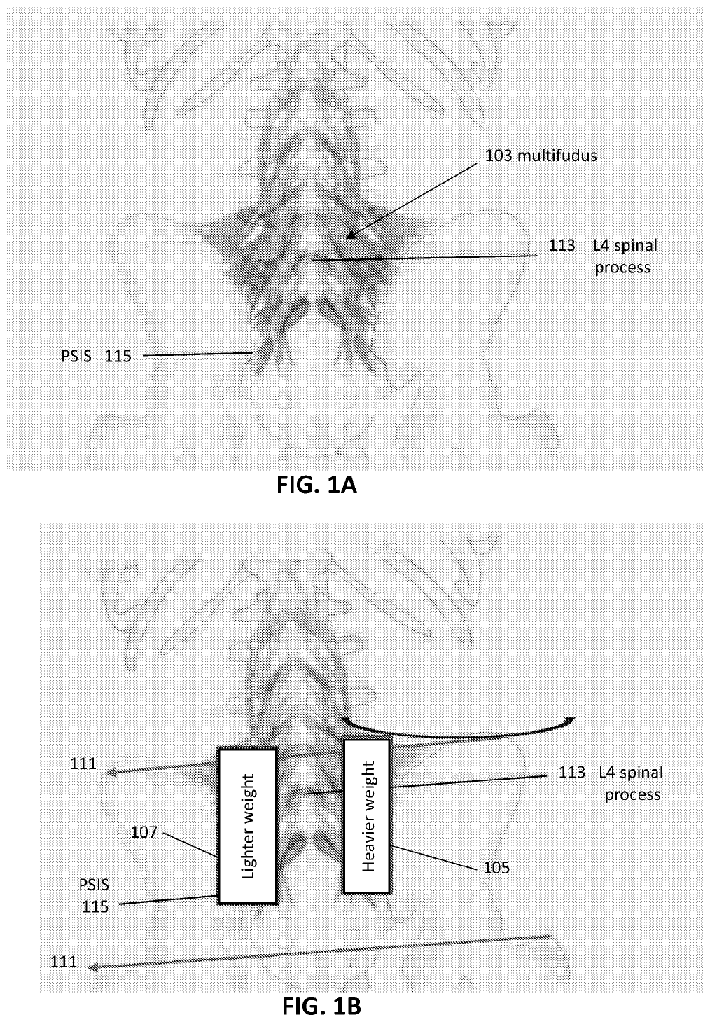

Enhancing just the spinal rotational stabilizers and extensors may allow the primary movers or large muscles, such as gluteus maximus, hip and back flexors, and extensors, to increase power immediately. For example, FIG. 1A illustrates the multifidi muscles 103, which are spinal stabilizers. There are multiple layers of spinal rotators that stabilize the spine and attach to spine, sacrum and pelvis. In FIG. 1A various spinal levels are shown including L1 to the bottom of the sacrum (L4 is shown 113 in this example). The posterior Iliac spinous process (PSIS) is also illustrated. In some cases the light weighting may be applied on either side of the subject's spine in the region between the subject's first lumbar vertebra (L1) and a bottom of the subject's sacrum level (S5), and laterally on the same spinal level, e.g., about or between the posterior edges of the subject's iliac crest, or between the lateral edges of the pelvis, etc. (e.g., between 0.5 and 8 inches on either side of the patient's midline/spine, between 0.5 and 7 inches on either side of the patient's midline/spine, between 0.5 and 6 inches on either side of the patient's midline/spine, between 0.6 and 5 inches on either side of the patient's midline/spine, between 0.5 and 4 inches on either side of the patient's midline/spine, between 0.5 and 3.5 inches on either side of the patient's midline/spine, between 0.5 and 3 inches on either side of the patient's midline/spine, etc.).

FIG. 1B schematically illustrates the positioning of a pair of asymmetric weights on either side of a subject's spine in a subject having a rotational deficit. FIG. 1B shows a view of a garment (e.g., shorts) and placement of asymmetric weights over the anatomy. These sensory inputs could be placed in any garment; belt, underwear, tights, etc., an in particular garments having a waist (“waisted region”) that may secure the stimulator against the subject. In FIG. 1B, the subject may have a Left Trendelenburg or left rotational weakness, which may be identified manually or automatically, as described below. In this example, the spinal levels are angled 111 relative to the horizontal and asymmetric weights (e.g., weights having different values) are positioned on either side (symmetrically) of the subject's spine by wearing a garment including the weights 105, 107. The weights 105, 107 may be attached or coupled to the garment either permanently or removably. In this example, the heavier weight (e.g., 1.5 pounds to ¼ pound, such as ½ pound) is attached to or integral to the garment (not shown) and is positioned on the weaker right side of the pelvis when the garment is worn by the subject. The second (lighter) weight 107 (e.g., 1 pound to ⅛ pound, such as ¼ pound) is attached to or integral to the garment. The garment may be shorts, pants, underwear, a belt, etc. When worn by the subject, the weights cover the spinal rotators and the PSIS, and the heavier 105 and lighter 107 weights are positioned an equal distance from the midline/center of the spine. Although FIG. 1B shows an example in which the stimulator is a weight, in some examples the stimulator may instead or additionally be an electronic stimulator, vibrational stimulator, etc.

The stimulator coupled to the garment may be coupled in any appropriate manner. For example the stimulator (e.g., weights) that is placed over the rotators and PSIS could be weights placed or attached to, or on, or in pockets, panels, etc. spaced apart (e.g., by between 0.1 and 20 cm apart), as illustrated in FIG. 1B. The weights could be attached to a panel by any means and applied by any means to the garment. The weights themselves can be made of a flexible weighted material that is part of the garment and/or sewn in. These weights could be made of silicon and a metal powder, or the weights could simultaneously be a fabric that has a weight combined within the fabric. Flexible weighted material could attach in any way to the shorts; they could be moved up and down in the shorts to enable the exact location required to stimulate improvement in rotational control of the pelvis. The garment may be any appropriate garment, such as any short, underwear or tights/pants, etc.

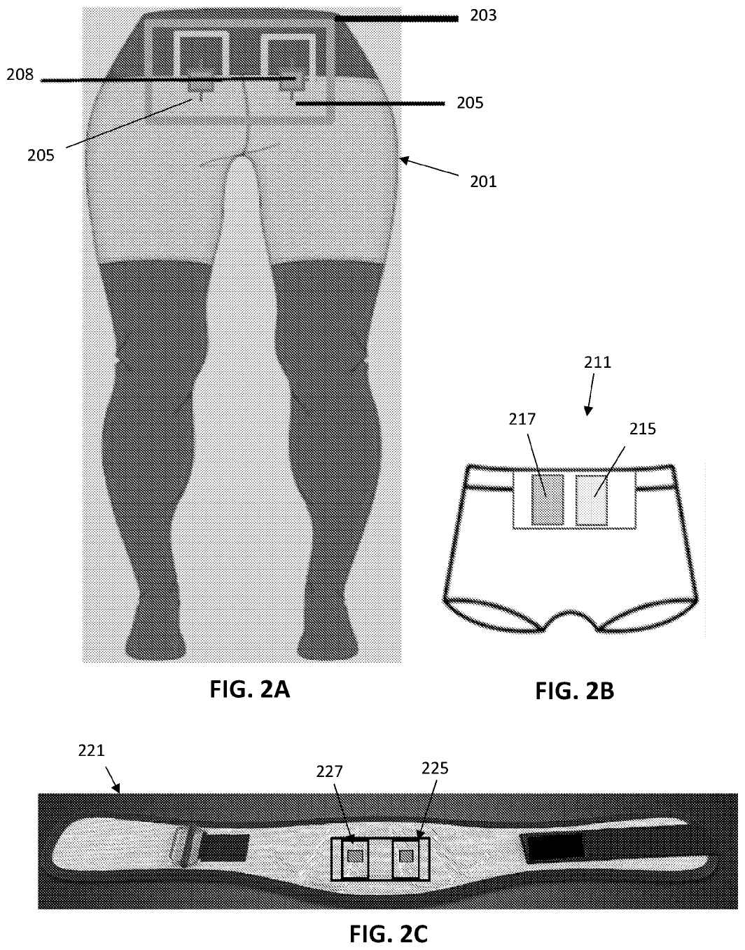

FIGS. 2A-2C show examples of garments 201, 211, 221 as described herein configured as a pair of shorts (FIG. 2A) and as a belt (FIG. 2C) that could be used, e.g., for weightlifting or therapy. Symmetrically positioned asymmetric weights (e.g., asymmetrical stimulus) may be placed on the inside of the belt. Optionally, other weights can be added in different patterns on the outside. In FIG. 2A the garment 201 include a single compound weight formed by a weighted panel 203 that includes a pair of weights 205, 207 that are positioned in this example over the subject's PSIS 208. FIG. 2B shows an example of a garment that is configured as an undergarment (underwear) 211 including a weighted panel (e.g., a compound weight) with a first weight 215 that is heavier than the second weight 217. The first and second weights 215, 217 are configured to be positioned on either side of the spine (midline), as described herein. In FIG. 2C the belt includes a pair of asymmetrically weighted weights 225, 227 that are positioned on the garment (belt 221) so as to be worn over the subject's PSIS region.

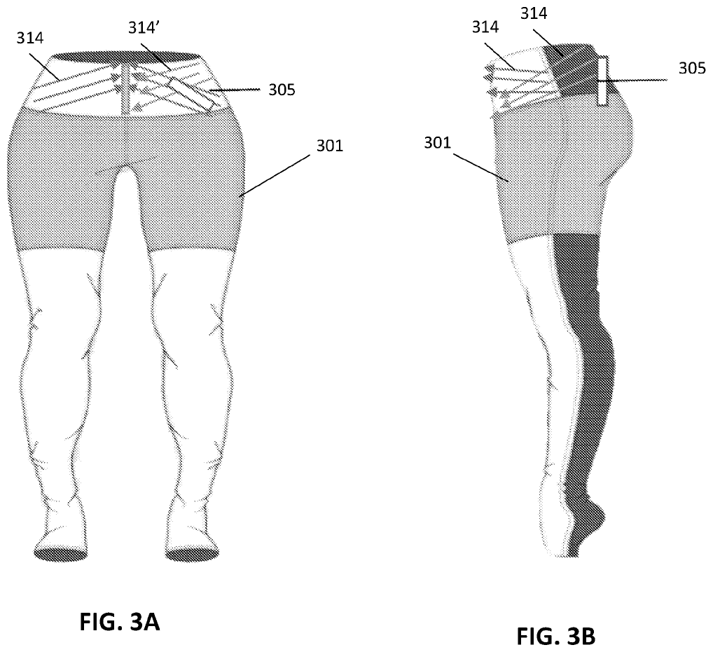

Any of these garments may include additional supports (e.g., bands, compression fabric, etc.) to secure the garment and the stimulator (e.g., weight) in position. For example, FIGS. 3A-3B illustrate another example of a garment configured as shorts or undergarments 301 that include one or a pair of weights 305 configured to be positioned on either side of the subject's spine in the L1 to bottom of the sacrum region in this example. The shorts also include supports 314, 314′ that are configured to apply a force to the garment when worn by the subject. In FIG. 3A, the supports 314, 314′ apply a pull force on the fabric; this pull force may be the result of a material, such as a panel of fabric attached by any means to the inside or outside of the garment. In FIGS. 3A and 3B the pull force may engage the internal obliques, a primary stabilizer of the torso. The internal oblique attaches to the crest of the ilium, and its action pulls the body forward, and provides ipsilateral rotation and lateral flexion control. The ilium is part of the pelvis and may require asymmetrical facilitation to enhance pelvis stabilization coordinating pelvic floor relaxation and tone. FIG. 3A illustrates one example of a garment that enables a lateral and anterior diagonal placement for a weighted stimulus and an upward pull or downward pull to assist in facilitating the lower abdominal.

To further facilitate stabilization of the core, weights or other stimuli can be placed over, under, within the garment in any appropriate manner. For example, a panel may be configured to provide additional pull to further enhance compression at the waist or to maintain position of the stimuli. FIG. 3B illustrates a pull force from the back of the garment that compresses the PSIS and low back extensor which may or may not contain asymmetrically placed stimuli in addition to these pulls or stimuli place in the front or side of the garment. In FIGS. 3A and 3B a pulling force may be applied by the garment puling from the subject's back, under the navel. Pulling from the front may align with the internal obliques to support rotation and intra-abdominal pressure. The pulling fabric or structures (e.g., cords, elastic, etc.) may be sewn to the garment, e.g., into a center or via any other form of attachment to provide a diagonal based pull force pulling forward. In some examples the pulling force is directed from the side or back of the garment when worn, upward 314 or downward 314′ reaching.

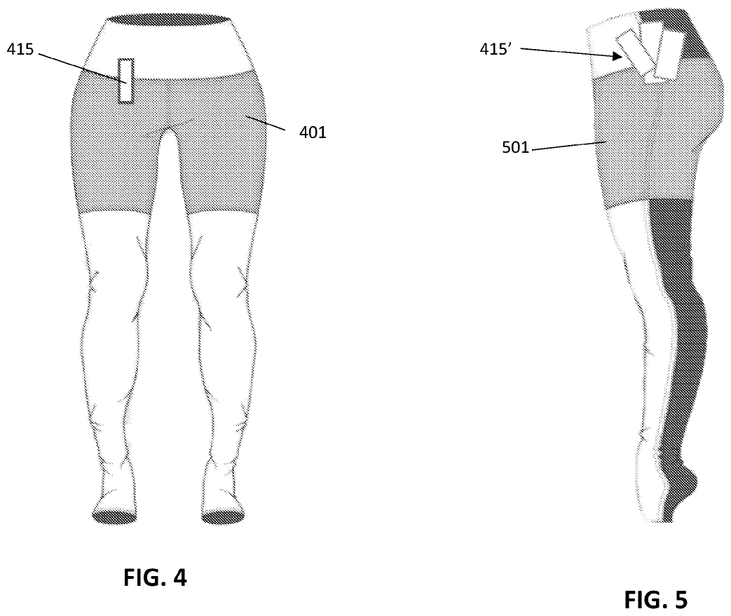

As mentioned above, any of these garments may also be configured to include additional components (e.g., weights, pads, etc.) in addition to the rotational correction weights (e.g., asymmetric stimulus to engage the subject's multifidi muscles on opposite sides of the subject's spine at approximately a same horizontal level). FIGS. 4 and 5 show examples of garments 401, 501 with such optional additional weights. In FIG. 4 the garment 401 includes an additional weight 415 for affecting the Ilio Psoas. In FIG. 5 the garment includes secondary weights 415′ configured to facilitate an input along the gluteus medius at angles that support the three anterior, medial, and posterior gluteus medius fibers. The garment may also contain any of the other features described above (e.g., asymmetric stimulus to engage the subject's multifidi muscles on opposite sides of the subject's spine at approximately a same horizontal level).

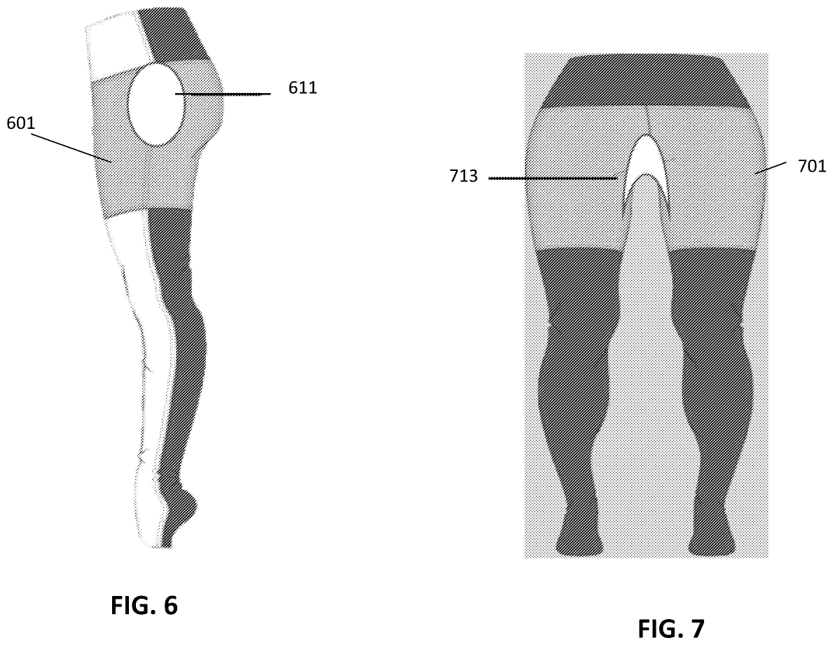

FIGS. 6 and 7 illustrate additional (optional) features that may be included in a garment as described herein. For example, in FIG. 6 the garment 601 includes a hip pad or silicone pad 611 for prevention of a hip fracture for people who may fall. In FIG. 7 the garment 701 includes an open crotch design to enable ease in use of sanitary facilities.

Any of the garments, e.g., configured as shorts or belts shown in FIGS. 2A-2C, 3A-3B, 4, 5, 6, and 7 may include a stimulator configured to apply an asymmetric stimulus (e.g., pair of weights) to engage the subject's multifidi muscles on opposite sides of the subject's spine at approximately a same horizontal level, and my also include a lower abdominal stabilizer to assist internal/external obliques and transverse abdominals and the back extensors for patients requiring lower back and lower abdominal activation creating intra-abdominal pressure.

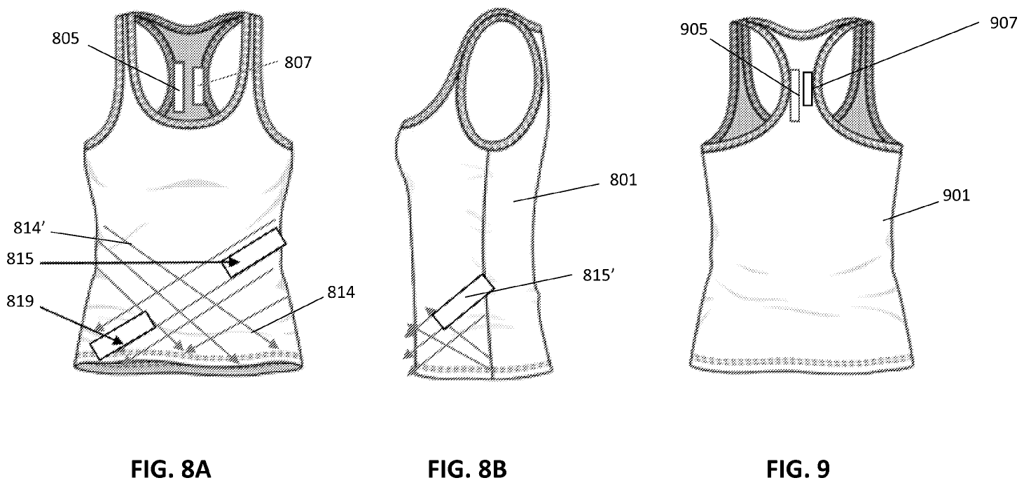

In some examples the garment may be configured as a shirt or may include a top (e.g., or bra). In some examples the garment may be configured to applying an asymmetric stimulus to engage the subject's multifidi muscles on opposite sides of the subject's spine at approximately a same horizontal level at a higher level (e.g., between T1 and T8) instead or in addition to between L1 and the bottom of the sacrum. For example, FIGS. 8A-8B and 9 illustrates garments 801, 901 configured as tops such as shirts or bras (e.g., a yoga shirt or bra). In FIG. 8A, the garment includes a pair of asymmetric weights 805, 807 configured to apply an asymmetric stimulus to engage the subject's multifidi muscles on opposite sides of the subject's spine at approximately a same horizontal level (between T1-T8). In this example, panels of stretchable fabric pattern may include pull elements (e.g., fibers) configured to apply force 814, 814′ along an internal and external oblique in the front and mid back area for the spinal rotators. These panels may be added afterward to the inside or outside of the garment. In addition, this region may be weighted 815, 819, as shown. In FIG. 9 the garment 901 includes a pair of asymmetric weights 905, 907 that are similarly positioned to those shown in FIGS. 8A-8B, but without any of the additional weights. In general any of the garments described herein (and methods of using them) may specifically exclude additional weights other than those applying an asymmetric stimulus to engage the subject's multifidi muscles on opposite sides of the subject's spine at approximately a same horizontal level.

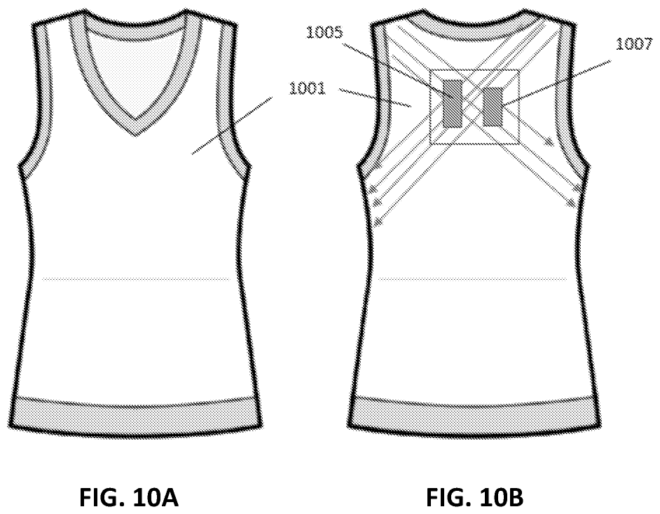

Similarly, FIGS. 10A-10B illustrate another example of a garment 1001 configured as a top including a compound weight that has a pair of asymmetrically weighted weights 1005, 1007 on the back in a region over the T1-T8 region. The compound weight may be a single panel to which the individual weights are attached. The panel may be attached (e.g., sewn or removably secured) so that the first and second weights are spaced on either side of the subject's spine when the garment is worn. The garment may also include one or more panels or elements (e.g., pull elements) that apply a pull force across the back which may further secure the garment in position on the subject's back.

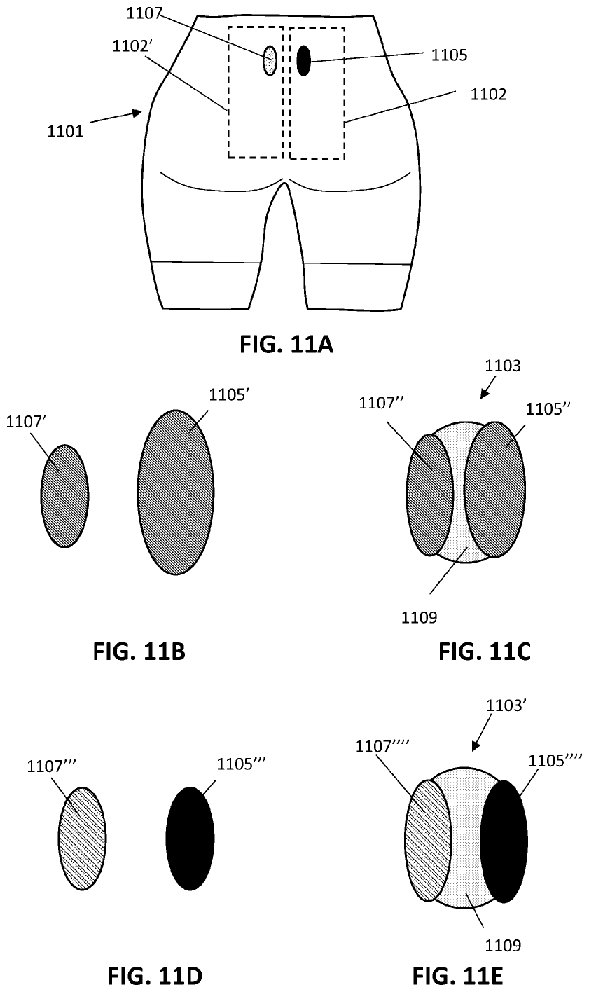

FIG. 11A illustrates another example of a garment configured as a pair of shorts 1101 to which a pair of asymmetrical stimulators 1105, 1107 are attached, each within a region 1102, 1102′ that is on either side of the subject's spine and between L1 and the bottom of the sacrum when the garment is worn. The first stimulator is configured to apply a grater intensity than the second stimulator. For example, the first stimulator and the second stimulator may both be weights and the first stimulator is heavier than the second stimulator. In some examples the first stimulator is an electrical stimulator that is delivering energy at a higher intensity than the second stimulator.

In FIG. 11A the stimulators may attached, e.g., by being sewn, etc. to the garment. Any appropriate stimulator(s) (e.g., weights, electrical stimulator, vibrational stimulator, etc.) may be attached to the garment symmetrically at the same level, but for delivery of asymmetric stimulation. FIGS. 11B-11E illustrate examples of different configurations of weights that may be used. In FIG. 11B the first weight 1105′ is heavier than the second weight 1107′; the two weights are separate and may be different sizes (but the same thickness, for example). The weights may be flexible or may be rigid. The weights may include a fabric backing and/or may be surrounded by fabric or padded. FIG. 11C shows another example of a set of weights 1105″, 1107″ that are configured as a compound weight 1103 both of which are attached (e.g., rigidly attached, in some cases adjustably attached) to a base 1109. The base may be attached to the garment at about the midline (e.g., along the spine of the patient) when the garment is worn and may be positioned at the appropriate level. In some examples the compound weight may be attached to the garment permanently or adjustably. The first and second weights may be positioned at a spaced-apart configuration (e.g., between 0.1 cm and 20 cm apart, etc.). In FIG. 11C the weights are shown as different sizes, with the heavier weight 1105″ being larger than the lighter weight 1107″. In FIG. 11D the two weights 1105′″, 1107′″ are different weights (e.g., 1105′″ is heavier than 1107′″) but are shown as the same size and/or shape. This may be beneficial for comfort and fit of the garment. FIG. 11E shows another example of a compound weight 1103′ in which the first, heavier, weight 1105″″ is the same size and shape as the second, lighter, weight 1107″″.

Although the weights shown in FIGS. 11A-11E are shown to be oval, any appropriate shape may be used, including round, rectangular, square, etc. the weights may be flexible, or rigid or may be segmented.

The first and second weights may be between about 0.05 pounds and 5 pounds (e.g., between 0.05 pounds and 3.5 pounds, etc.). In some cases the absolute weight may be less important than the difference between the lighter and the heavier weights (e.g., the percentage difference). For example, in some cases the light weight may be between 0.1% and 10% of the weight of the heavier weight (e.g., between 0.1% and 20%, between 1% and 25%, between 1% and 30%, between 1% and 40%, between 5% and 50%, between 5% and 60%, between 10% and 70%, between 10% and 75%, between 10% and 80%, between 10% and 85%, between 10% and 90%, between 10% and 95%, between 1% and 95%, between 0.1% and 95%, etc. of the weight of the heavier weight in a symmetric pair).

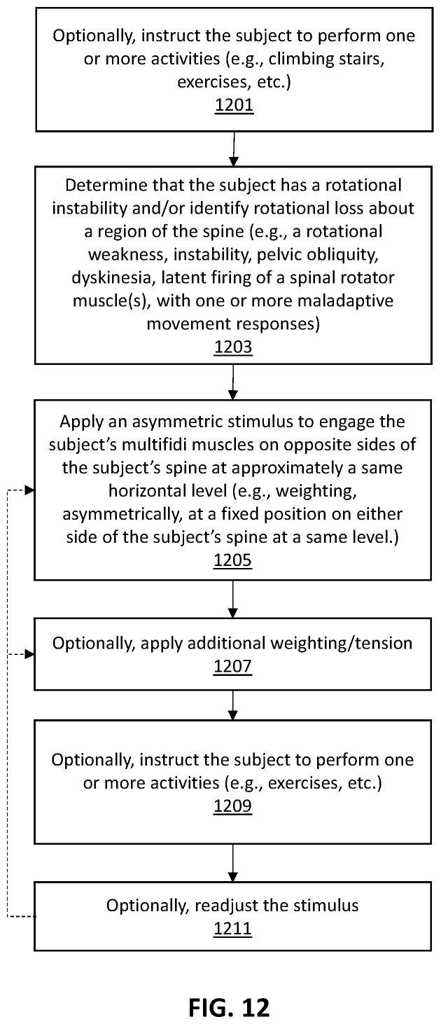

In practice, these apparatuses may be used as part of a method or with an apparatus (e.g., software, such as an app) for treating rotational deficit about a segment(s) of the subject's spine. FIG. 12 provides one schematic outline of a method as described herein. Any of these methods may include determining that the subject has a rotational instability and/or may include identifying rotational deficit about a region of the subject's spine. For example, a user skilled in the art of observing movement such as but not limited to a doctor, therapists, nurse, athletic trainer, Yoga instructor, strength/sport coach, personal trainer, etc. (or in some cases a software/firmware/hardware agent) may observe the subject to identify that they have a rotational instability and may provide a garment having an asymmetric stimulus as described herein. In any of these cases, the user (or agent) may instruct the subject to perform one or more movements or activities (e.g., maneuvers) such as climbing stairs, bending over, picking up an object (e.g., shoe), walking, etc. 1201 and may determine based on the movement (or generally based on an unscripted or undefined movement) that the subject has a rotational instability and/or rotational deficit 1203. Rotational instability and rotational deficit may be used interchangeably, herein. The user or agent may determine rotational instability and/or rotational deficit by detecting one or more of: a rotational weakness, instability, pelvic obliquity, dyskinesia, and/or latent firing of a spinal rotator muscle(s), with one or more maladaptive movement responses. The maladaptive movement may be, e.g., in the lower body which may include one or more of: pelvis drop, hip internal rotation and/or adduction, knee valgus, lower leg external rotation, ankle and/or foot pronation, great toe or all toes extension. The maladaptive movement may be in the upper body which may include one or more of: rib flare, trunk rotation and/or lateral flexion, scapular protraction and/or elevation, glenohumeral internal rotation, neck extension and/or rotation, forward head posture, and/or asymmetric arm swing during movement.

Once identified, the user or agent may apply (e.g., by instructing the patient to don a garment as described herein) an asymmetric stimulus to engage the subject's multifidi muscles on opposite sides of the subject's spine at approximately a same horizontal level 1205. The user or agent may determine the precise location of the asymmetric weights, including the spacing between the weights and/or the level at which the weights are attached to the garment (e.g., between L1 and the bottom of the sacrum, between T1-T8), and/or the amount of weight, based on the patient instability. For example, the user or agent may analyze the direction and amount of instability to determine which side of the spine to weight more heavily. In some cases if the subject's hips are dropping (e.g., angled) during a gross motor movement, the heavier weight may be positioned on the side that is lower. The level of the placement may be between L1 and the bottom of the sacrum generally, and more precise placement of the level may depend on how the subject's rotational instability presents to the user or agent. In some cases the user or agent may iteratively apply stimulation (e.g., weighting) by, e.g., applying asymmetric stimulus to engage the subject's multifidi muscles on opposite sides of the subject's spine at approximately at a first horizontal level 1205, then by observing the subject and/or asking the subject to perform a predetermined movement/activity 1209 and observing the subject. Based on the observed change in the rotational stability, the user or agent may readjust the stimulus, e.g., repositioning the stimulus (e.g., weight) and/or the intensity of the stimulus (e.g., weight, and/or additional electrical and/or vibrational stimulation) 1211. Optionally in any of these methods additional weighting and/or tension may be applied and adjusted 1207.

As mentioned, any of these methods and apparatuses may include software, firmware or hardware to perform these methods. For example, also described herein is software (e.g., a non-transitory computing device readable medium having instructions stored thereon that are executable by a processor to cause a computing device to perform any of these methods). Any of the apparatuses described herein may include a system having one or more processors, and a memory coupled to the one or more processors, the memory storing computer-program instructions, that, when executed by the one or more processors, perform a computer-implemented method such as the methods described above (and in FIG. 12).

For example, a computer-implemented method may be performed on a mobile computing device (e.g., phone), and may include recording the subject in motion, and/or instructing the user to have the subject, or directly instructing the subject, to perform one or more movements or maneuvers (e.g., compound movements) and may record (e.g., video) the subject moving. In some examples the apparatus (e.g., software) may further include one or more machine-learning agents that is trained on data of patients with and without rotational instability (rotational deficit) and in some cases, resulting effective weighting. The trained machine-learning agent (e.g., a neural network) may identify or determine a probability that the subject has a rotational instability. In some cases the machine learning agent may further recommend the application of asymmetric stimulus (e.g., weighting) including which garments (and/or where to apply stimulus/weight. Optionally the apparatus may further have the weighted (or otherwise treated) subject wearing the garment perform one or more additional movements/maneuvers and may further refine the applied asymmetric stimulus.

For example this apparatus (e.g., software, including an App) may be configured to take physical movement data (which may in some cases include landmarks on a garment, including the weighted or unweighted garments) and may estimate rotational balance deficit via an input, such as one or more of: video, motion capture, posture analysis, sensor, foot pressure, the center of mass, the center of gravity, the center of pressure or, other means, to determine the need for the input required to improve body symmetry via the use of asymmetric application of input.

As mentioned, the apparatus may automatically or semi-automatically detect abnormal motion and may recommend or suggest placement of asymmetric input applied to the body part. Examples of movements, such as predefined movements, maneuvers or activities that may be used to determine rotational instability may include movements of daily living such as narrow based stance positions, single leg movements such as stair climbing or a lunge which may reveal maladaptive movement compensations in the upper or lower body. For example, maladaptive movements in the lower body may include: pelvis drop, hip internal rotation and/or adduction, knee valgus, lower leg external rotation, ankle and/or foot pronation, great toe or all toes extension. Maladaptive movements in the upper body may include one or more of the following: rib flare, trunk rotation and/or lateral flexion, scapular protraction and/or elevation, glenohumeral internal rotation, neck extension and/or rotation, forward head posture, asymmetric arm swing during movement. Analyzing these movements may be used as part of these methods and apparatuses and may include recorded (e.g., via video, COP, COM, etc.) in order to identify that a subject has a rotational deficit. In some examples the method or apparatus may include a gravity eliminated analysis, and/or a changeable surfaces analysis.

Thus, in general, these methods and apparatuses may use a correlation between rotational weakness and pelvic obliquity, dyskinesia or incoordination, and latent firing at the spinal rotators to apply an appropriate light asymmetric weighted sensory input to enhance control of rotation. Input to the muscular rotators in the posterior aspect of the lower back (e.g., the multifidi muscles) may affect anterior, lateral, posterior, and rotational insufficiency of the upper and lower torso. Stabilization of the rotators improves pelvic obliquity, dyskinesia, incoordination, muscular firing patterns, posture, and symmetry of the body. Similarly, the upper torso may do the same. Input to the anterolateral muscles of the upper or lower back can also improve rotational symmetry.

For example, described herein are method and apparatuses for the strategic application of weight or any muscular or nervous system stimulating device or devices to one or more locations on one's torso. In general, one of the weight or devices may be heavier or provide differential input to the other side and is generally referred to herein as asymmetric weighting. Individual weights may provide differential input. Since pairs of differently weighted weights may be used, these pairs may be combined together to form a compound weight. For example, an asymmetrically weighted input may be contained within a continuous weight. In some examples an asymmetric set of weights may be sewn, or attached by any appropriate method into a panel, which may be waterproof, and/or into a garment (e.g., undergarments, pants, shorts, belt, sash, sports bra, Yoga top, etc.). The pair of asymmetric weights may be horizontally symmetrically positioned, e.g., and may be configured to place sensory input to the vertical axis. In some examples the apparatus may be configured so that vertical movement of the stimulation (e.g., weights) is not fixed, but may be adjusted and/or later secured or fixed. Weight placement in the lower torso may be arranged so as to not cross the sacral midline.

In general, as mentioned above, the asymmetrically weighted weight made of any material substance in any form (e.g., shape, size, etc.) at the weights described herein (e.g., ½-¼, ⅓-⅙, ¼-⅛, ⅛- 1/16th and 1/16- 1/32^{nd} pounds, or heavier).

For example, an asymmetrical application of weight may be supplied to an area of the body identified a need of stimulation to enhance nanobalance by facilitating the uptake to the nervous system and musculature under the input, providing proprioceptive, contractile, stretch, pull, pressure, or other means of sensory conduction. The stimulation may be applied to the area of the spinal musculature (e.g., spinal column, sacrum, etc.) as described herein. The stimulation may be applied to musculature with unequal pull and/or may be applied diagonally on opposite sides of the body bilaterally.

In general, the apparatuses described herein may be reversible (e.g., a pair of reversible underwear or reversible shorts) so that they have an asymmetrical pattern of weighting and/or other stimulator providing different output or input as described herein, in order to assist in the application of asymmetric input to a posterior aspect of the back for the treatment of a variety of individuals who have different rotation weaknesses. Reversible garments may allow heavier (asymmetric) weighting of one side of the other by turning the shorts inside out.

As mentioned, any of these methods and apparatuses (e.g., app software) may include identifying a rotational loss about a segment of the spine, including identifying by a functional evaluation that may define the asymmetrical loss across the spine. Rotational deficit may be treated at the subject's pelvis first because it may be closest to the body's center of gravity and depending on how the body has been compensating there may also be rotational loss further up the spine. As the body's rotational weakness becomes greater, there may be less stability at the torso's center of gravity. The body may then re-establish core stability. For example, a Valsalva maneuver may create core stability (e.g., diaphragmatic stability patterning seen with a rib flare). Another example is the upper trap and neck stabilizers which may utilize the weight of the head to balance the center of gravity which may be seen with excessive neck extension, and/or head rotation, and/or forward head posture. Additionally, distal stability may be increased through the lower limb or upper limb via co-contraction patterns at the distal large joints. In the lower body, the ankle for example, may utilize anterior tibialas and soleus co-contraction patterns seen with great toe extension, or extension of the toes in stance. This lower stability pattern may create a longer stance phase with the ground and may create greater stability at the body's center of gravity. In the upper body, the shoulder for example, may utilize the upper trapezius and pectoralis minor co-contraction pattern seen with glenohumeral internal rotation and/or scapular protraction, and/or scapular elevation and/or asymmetrical arm swing with movement.

As mentioned, the asymmetric weighting may be configured so that the change in weights between the pairs of symmetrically positioned weights may be about, e.g., 50%, but could be greater or lesser. The weighting may be positioned symmetrically at a fixed distance away from central spine equally on both sides; the distance away from spine may be determined by the level of rotational deficit.

### Examples

The methods and apparatuses described herein have been successfully used to provide an increase in athletic performance. A first subject was a female rower. The second subject was a female weightlifter. Placing the weights over the multifidi of each weightlifter/rower that wasn't functioning at peak performance (rotation was not stable) and a lighter weight over the opposite PSIS increased a lift from 100 pounds to 125 pounds in a single session, representing a remarkable 25% improvement single-session. Typical increases in single-session improvement are about 5% or a few pounds, thus a 25% increase in the amount of weight able to be lifted same-session by the rower/weightlifters was extraordinary and unexpected. The rower and weightlifter each reported they could not increase their lift beyond 100 pounds for six weeks prior. Further, their primary mover muscles and ligaments did not have the usual fatigue and muscle soreness for an increased lift.

Similar results have been found for treating incontinence. One subject, who had reported having to get up every night to urinate for seven years did not have to after the first weighing application. The subject also reported improvement in pelvic heaviness and reduced back pain in sitting or standing. A different subject trialing this new weighted adaptation claimed that after six weeks, her urinary leakage was gone, and she reclaimed her functional ability to climb stairs. These reports suggest that the multifidi attachment into the sacrum was likely playing a role in stabilizing the pelvis, pelvic floor, and back providing stability to improve ability.

It should be appreciated that all combinations of the foregoing concepts and additional concepts discussed in greater detail below (provided such concepts are not mutually inconsistent) are contemplated as being part of the inventive subject matter disclosed herein and may be used to achieve the benefits described herein.

The process parameters and sequence of steps described and/or illustrated herein are given by way of example only and can be varied as desired. For example, while the steps illustrated and/or described herein may be shown or discussed in a particular order, these steps do not necessarily need to be performed in the order illustrated or discussed. The various example methods described and/or illustrated herein may also omit one or more of the steps described or illustrated herein or include additional steps in addition to those disclosed.

Any of the methods (including user interfaces) described herein may be implemented as software, hardware or firmware, and may be described as a non-transitory computer-readable storage medium storing a set of instructions capable of being executed by a processor (e.g., computer, tablet, smartphone, etc.), that when executed by the processor causes the processor to control perform any of the steps, including but not limited to: displaying, communicating with the user, analyzing, modifying parameters (including timing, frequency, intensity, etc.), determining, alerting, or the like. For example, any of the methods described herein may be performed, at least in part, by an apparatus including one or more processors having a memory storing a non-transitory computer-readable storage medium storing a set of instructions for the processes(s) of the method.

While various embodiments have been described and/or illustrated herein in the context of fully functional computing systems, one or more of these example embodiments may be distributed as a program product in a variety of forms, regardless of the particular type of computer-readable media used to actually carry out the distribution. The embodiments disclosed herein may also be implemented using software modules that perform certain tasks. These software modules may include script, batch, or other executable files that may be stored on a computer-readable storage medium or in a computing system. In some embodiments, these software modules may configure a computing system to perform one or more of the example embodiments disclosed herein.

As described herein, the computing devices and systems described and/or illustrated herein broadly represent any type or form of computing device or system capable of executing computer-readable instructions, such as those contained within the modules described herein. In their most basic configuration, these computing device(s) may each comprise at least one memory device and at least one physical processor.

The term “memory” or “memory device,” as used herein, generally represents any type or form of volatile or non-volatile storage device or medium capable of storing data and/or computer-readable instructions. In one example, a memory device may store, load, and/or maintain one or more of the modules described herein. Examples of memory devices comprise, without limitation, Random Access Memory (RAM), Read Only Memory (ROM), flash memory, Hard Disk Drives (HDDs), Solid-State Drives (SSDs), optical disk drives, caches, variations or combinations of one or more of the same, or any other suitable storage memory.

In addition, the term “processor” or “physical processor,” as used herein, generally refers to any type or form of hardware-implemented processing unit capable of interpreting and/or executing computer-readable instructions. In one example, a physical processor may access and/or modify one or more modules stored in the above-described memory device. Examples of physical processors comprise, without limitation, microprocessors, microcontrollers, Central Processing Units (CPUs), Field-Programmable Gate Arrays (FPGAs) that implement softcore processors, Application-Specific Integrated Circuits (ASICs), portions of one or more of the same, variations or combinations of one or more of the same, or any other suitable physical processor.

Although illustrated as separate elements, the method steps described and/or illustrated herein may represent portions of a single application. In addition, in some embodiments one or more of these steps may represent or correspond to one or more software applications or programs that, when executed by a computing device, may cause the computing device to perform one or more tasks, such as the method step.

In addition, one or more of the devices described herein may transform data, physical devices, and/or representations of physical devices from one form to another. Additionally or alternatively, one or more of the modules recited herein may transform a processor, volatile memory, non-volatile memory, and/or any other portion of a physical computing device from one form of computing device to another form of computing device by executing on the computing device, storing data on the computing device, and/or otherwise interacting with the computing device.

The term “computer-readable medium,” as used herein, generally refers to any form of device, carrier, or medium capable of storing or carrying computer-readable instructions. Examples of computer-readable media comprise, without limitation, transmission-type media, such as carrier waves, and non-transitory-type media, such as magnetic-storage media (e.g., hard disk drives, tape drives, and floppy disks), optical-storage media (e.g., Compact Disks (CDs), Digital Video Disks (DVDs), and BLU-RAY disks), electronic-storage media (e.g., solid-state drives and flash media), and other distribution systems.

A person of ordinary skill in the art will recognize that any process or method disclosed herein can be modified in many ways. The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed.

The various exemplary methods described and/or illustrated herein may also omit one or more of the steps described or illustrated herein or comprise additional steps in addition to those disclosed. Further, a step of any method as disclosed herein can be combined with any one or more steps of any other method as disclosed herein.

The processor as described herein can be configured to perform one or more steps of any method disclosed herein. Alternatively or in combination, the processor can be configured to combine one or more steps of one or more methods as disclosed herein.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

In general, any of the apparatuses and methods described herein should be understood to be inclusive, but all or a sub-set of the components and/or steps may alternatively be exclusive and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1-30. (canceled)

31. A garment for treating rotational deficit about one or more segments of a subject's spine, the garment comprising:
a body region configured to be worn by the subject; and
a first weight and a second weight, wherein the first weight is greater than the second weight, further wherein the first and second weights are symmetrically coupled to the body region and configured to be positioned on the subject's body between either the subject's first thoracic vertebra (T1) and the subject's eighth thoracic vertebra (T8) or between the subject's first lumbar vertebra (L1) and a bottom of the subject's sacrum level (S5) symmetrically on either side of the patient's spine so that the subject is asymmetrically weighted when the garment is worn by the subject.

31. The garment of claim 31, wherein the body region is configured to secure the first weight and the second weight against the subject's skin when the garment is worn.

33. The garment of claim 31, wherein the first weight and the second weight are separated from each other by between about 0.5 cm and 20 cm.

34. The garment of claim 31, wherein the garment is reversible.

35. The garment of claim 31, configured as a pair of shorts, an undergarment, or a belt, wherein the first and second weights are configured to be positioned on the subject's body between the subject's first lumbar vertebra (L1) and the bottom of the subject's sacrum level (S5) symmetrically on either side of the patient's spine when the garment is worn by the subject.

36. The garment of claim 31, configured as a bra or an undergarment, wherein the first and second weights are configured to be positioned on the subject's body between the subject's first thoracic vertebra (T1) and the subject's eighth thoracic vertebra (T8) symmetrically on either side of the patient's spine when the garment is worn by the subject.

37. The garment of claim 31, wherein the first weight is between 1/16 pound and 5 pounds and wherein the second weight is between 1/32 and 5 pounds.

38. The garment of claim 31, wherein the first weight and the second weight are part of a compound weight attached to the garment.

39. The garment of claim 31, further comprising an electric stimulator or vibrational stimulator positioned with either the first weight or the second weight, or a first electrical or vibrational stimulator positioned with the first weight and a second electrical or vibrational stimulator positioned with the second weight.

40. A garment for treating rotational deficit about one or more segments of the subject's spine, the garment comprising:
a waisted region configured to be worn by a subject; and
a first weight and a second weight, wherein the first weight is greater than the second weight, further wherein the first and second weights are each coupled to the torso region and configured to be positioned on the subject's body between the subject's first lumbar vertebra (L1) and a bottom of the subject's sacrum level (S5) symmetrically on either side of the patient's spine so that the subject is asymmetrically weighted when the garment is worn by the subject.

41. The garment of claim 40, wherein the first weight and the second weight are separated from each other by between about 0.5 cm and 20 cm.

42. The garment of claim 40, wherein the garment is reversible.

43. The garment of claim 40, wherein the first weight is between 1/16 pound and 5 pounds and wherein the second weight is between 1/32 and 5 pounds.

44. The garment of claim 40, wherein the first weight and the second weight are part of a compound weight attached to the garment.

45. The garment of claim 40, further comprising an electric stimulator or vibrational stimulator positioned with either the first weight or the second weight, or a first electrical or vibrational stimulator positioned with the first weight and a second electrical or vibrational stimulator positioned with the second weight.

46. A weighted garment for treating rotational deficit about one or more segments of the subject's spine, the garment comprising:
a torso region configured to be worn by a subject; and
a first weight and a second weight, wherein the first weight is greater than the second weight, further wherein the first and second weights are each coupled to the torso region and configured to be positioned on the subject's body between the subject's first thoracic vertebra (T1) and the subject's eighth thoracic vertebra (T8) symmetrically on either side of the patient's spine so that the subject is asymmetrically weighted when the garment is worn by the subject.

47. The garment of claim 46, wherein the first weight and the second weight are separated from each other by between about 0.5 cm and 20 cm.

48. The garment of claim 46, wherein the garment is reversible.

49. The garment of claim 46, configured as a bra or an undergarment.

50. The garment of claim 46, wherein the second weight is between 1/32 and 3 pounds and the first weight is between ⅛ and 5 pounds.

51. The garment of claim 46, wherein the first weight and the second weight are part of a compound weight.
